(19) United States
(12) Patent Application Publication
(10) Pub. No.: US 2024/0289948 A1
(43) Pub. Date: Aug. 29, 2024

(54) IMAGE ANALYSIS METHOD AND IMAGE ANALYSIS SYSTEM

(71) Applicant: InnoCare Optoelectronics Corporation, Tainan City (TW)

(72) Inventors: Jing-Yao Wang, Tainan City (TW); Yu-Heing Chen, Tainan City (TW)

(73) Assignee: InnoCare Optoelectronics Corporation, Tainan City (TW)

(21) Appl. No.: 18/402,749

(22) Filed: Jan. 3, 2024

(30) Foreign Application Priority Data
Feb. 20, 2023 (TW) 112106058

(51) Int. Cl. G06T 7/00; G16H 50/20

(52) U.S. Cl. CPC G06T 7/0012; G16H 50/20; G06T 2207/10116; G06T 2207/20076; G06T 2207/20084; G06T 2207/30096

(57) ABSTRACT

An image analysis method and an image analysis system are provided. The image analysis system includes an X-ray sensor, a computing device and a display device. The computing device is coupled to the X-ray sensor. The computing device includes a processing module and a memory module. The display device is coupled to the computing device. The processing module executes an image processing unit and an image analysis unit stored in the memory module to perform an image analysis according to dual-energy image data generated by the X-ray sensor, and outputs a lesion judgment result to the display device.

### CROSS-REFERENCE TO RELATED APPLICATION

This application claims the priority benefit of Taiwan application Ser. No. 112106058, filed on Feb. 20, 2023. The entirety of the above-mentioned patent application is hereby incorporated by reference herein and made a part of this specification.

### BACKGROUND

### Technical Field

The disclosure relates to an image processing technology, and in particular relates to an image analysis method and an image analysis system.

### Description of Related Art

Currently, the medical image used to analyze whether a lesion is present is a single-energy X-ray image generated by a single-exposure sensing by a general X-ray sensor. Therefore, due to the limited image information in the single-energy X-ray image, the conventional lesion judgment has the problem of poor accuracy.

### SUMMARY

The disclosure provides an image analysis method and an image analysis system, which may effectively analyze and process dual-energy image data, so as to effectively determine whether lesions appear in medical images.

The image analysis method of this disclosure includes the following operation. Dual-energy image data is obtained. A standard image, a soft tissue image, and a hard tissue image are generated according to the dual-energy image data. A first image analysis is performed on the standard image to generate a first lesion probability value. Whether the first lesion probability value is higher than a first threshold is determined. When the first lesion probability value is lower than or equal to the first threshold, a second image analysis is performed on at least one of the soft tissue image and the hard tissue image to generate a second lesion probability value. Whether the second lesion probability value is higher than a second threshold is determined. When the second lesion probability value is higher than the second threshold, a lesion judgment result is output.

The image analysis method of this disclosure includes the following operation. Dual-energy image data is obtained. At least one of a first image and a second image is generated according to the dual-energy image data. Image segmentation is performed on the first image to generate a mask image. The first image and the mask image are combined, or the second image and the mask image are combined, to generate a combined mask image. Image analysis is performed on the combined mask image to generate a third lesion probability value. When the third lesion probability value is higher than a third threshold, a lesion judgment result is output.

The image analysis system disclosed in the disclosure includes an X-ray sensor, a computing device, and a display device. The computing device is coupled to the X-ray sensor. The computing device includes a processing module and a memory module. The processing module executes an image processing unit and an image analysis unit stored in the memory module to perform an image analysis according to dual-energy image data generated by the X-ray sensor, and output a lesion judgment result to the display device.

Based on the above, the image analysis method and image analysis system of this disclosure may generate multiple medical images based on the dual-energy image data obtained by the X-ray sensor, and perform image analysis based on these medical images to determine whether lesions are present in these medical images, so as to help medical personnel to efficiently determine whether there is a potential risk of disease.

In order to make the aforementioned features and advantages of the disclosure comprehensible, embodiments accompanied with drawings are described in detail below.

### BRIEF DESCRIPTION OF THE DRAWINGS

FIG. 1 is a schematic diagram of an image analysis system according to an embodiment of the disclosure.

FIG. 2 is a flowchart of an image analysis method according to an embodiment of the disclosure.

FIG. 3A is a schematic diagram of a standard image according to an embodiment of the disclosure.

FIG. 3B is a schematic diagram of a soft tissue image according to an embodiment of the disclosure.

FIG. 3C is a schematic diagram of a hard tissue image according to an embodiment of the disclosure.

FIG. 4 is a flowchart of generating a second lesion probability value according to an embodiment of the disclosure.

FIG. 5 is a flowchart of generating a second lesion probability value according to another embodiment of the disclosure.

FIG. 6 is a flowchart of an image analysis method according to another embodiment of the disclosure.

FIG. 7 is a schematic diagram of a mask image according to an embodiment of the disclosure.

### DETAILED DESCRIPTION OF DISCLOSED EMBODIMENTS

References of the exemplary embodiments of the disclosure are to be made in detail. Examples of the exemplary embodiments are illustrated in the drawings. If applicable, the same reference numerals in the drawings and the descriptions indicate the same or similar parts.

Certain terms may be used throughout the disclosure and the appended patent claims to refer to specific elements. It should be understood by those skilled in the art that electronic device manufacturers may refer to the same components by different names. The disclosure does not intend to distinguish between components that have the same function but have different names. In the following description and patent claims, words such as “comprising” and “including” are open-ended words, so they should be interpreted as meaning “including but not limited to . . . ”.

In this disclosure, terms related to joining and connecting, such as “connected”, “interconnected”, etc., unless otherwise defined, may mean that two structures are in direct contact, or may also mean that two structures are not in direct contact, in which there are other structures located between these two structures. The terms related to joining and connecting may also include the case where both structures are movable, or both structures are fixed. Furthermore, the term “coupled” includes any direct or indirect means of electrical connection. In the case of a direct electrical connection, the end points of two elements on a circuit directly connect to each other, or connect to each other through a conductive wire. In the case of indirect electrical connection, a switch, a diode, a capacitor, an inductor, a resistor, other suitable elements, or a combination thereof, but not limited therein, is between the end points of two elements on a circuit.

In this disclosure, the terms “about”, “equal to”, “equal” or “same”, “substantially” or “generally” are interpreted as within 20% of a given value or range, or interpreted as within 10%, 5%, 3%, 2%, 1%, or 0.5% of the given value or range.

In this disclosure, any two values or directions used for comparison may have certain errors. Furthermore, the terms “a given range is from a first value to a second value”, “a given range is within a range from the first value to the second value” means that the given range includes the first value, the second value, and other values in between.

In this disclosure, the terms such as “first”, “second”, etc. used in the description and the patent claims are used to modify elements, which do not imply and represent that the, or these, components have any previous ordinal numbers, and also does not represent the order of a certain element and another element, or the order of the manufacturing method. The use of these ordinal numbers is to only clearly distinguish an element with a certain name from another element with the same name. The same terms may not be used in the patent claims and the description, and accordingly, the first component in the description may be the second component in the patent claims. It should be noted that, in the following embodiments, the technical features in several different embodiments may be replaced, reorganized, and mixed to complete other embodiments without departing from the spirit of the disclosure.

It should be noted that, in the following embodiments, the features in several different embodiments may be replaced, reorganized, and mixed to complete other embodiments without departing from the spirit of the disclosure. As long as the features of the various embodiments do not violate the spirit of the disclosure or conflict with one another, they may be mixed and matched arbitrarily.

Unless otherwise defined, all terms (including technical and scientific terms) used herein have the same meaning as commonly understood by one of ordinary skill in the art to which this disclosure belongs. It is understood that these terms, such as those defined in commonly used dictionaries, should be interpreted as having meanings consistent with the relevant art and the background or context of the disclosure, and should not be interpreted in an idealized or overly formal manner, unless otherwise defined in the embodiments of the disclosure.

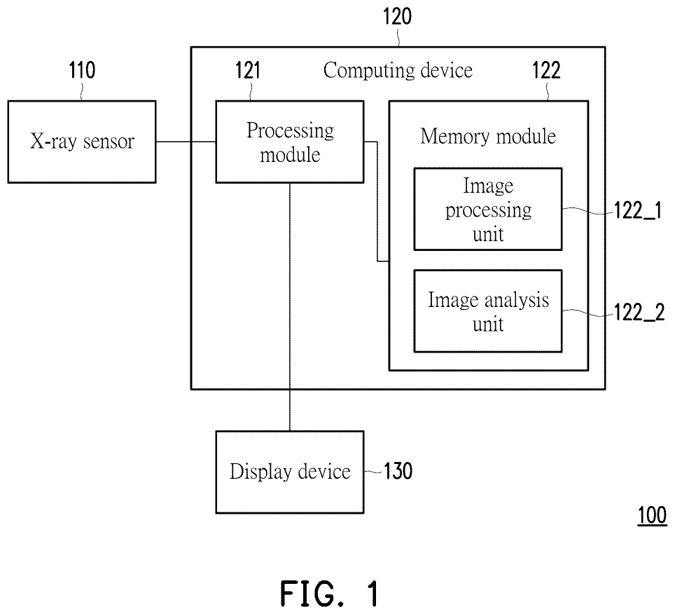

FIG. 1 is a schematic diagram of an image analysis system according to an embodiment of the disclosure. Referring to FIG. 1, an image analysis system 100 includes an X-ray sensor 110, a computing device 120, and a display device 130. The computing device 120 is coupled to the X-ray sensor 110 and the display device 130. The computing device 120 includes a processing module 121 and a memory module 122. The processing module 121 is coupled to the memory module 122, and is coupled to the X-ray sensor 110 and the display device 130. The memory module 122 includes an image processing unit 122_1 and an image analysis unit 122_2.

In this embodiment, the computing device 120 may be a device such as a personal computer (PC), a laptop, a tablet, or a smart phone. The processing module 121 of the computing device 120 may output according to user control or automatically output control signals to the X-ray sensor 110, and may provide image data to the display device 130. In this embodiment, the X-ray sensor 110 may be a flat panel detector, and is connected to the computing device 120 in a wired or wireless manner. Alternatively, in an embodiment, the X-ray sensor 110 and the computing device 120 may be integrated into a flat panel sensor device. In addition, the computing device 120 and the display device 130 may be integrated into a single electronic device, or the computing device 120 and the display device 130 may also be two separate devices that are connected in a wired or wireless manner.

In this embodiment, the processing module 121 may include a processor, and the processor may be, for example, a field programmable gate array (FPGA) or a graphics processing unit (GPU), or other suitable elements, and the disclosure is not limited thereto. The memory module 122 may include a memory, and may store the image processing unit 122_1 and the image analysis unit 122_2. The memory may be, for example, a dynamic random-access memory (DRAM) or a non-volatile memory (NVM), etc., and the disclosure is not limited thereto. In this embodiment, the processor may be used to execute the image processing unit 122_1 and the image analysis unit 122_2 stored in the memory, and may also store the image data in the memory.

In this embodiment, the processing module 121 may execute the image processing unit 122_1 and the image analysis unit 122_2 stored in the memory module 122 to perform image analysis according to the dual-energy image data generated by the X-ray sensor 110, and output the lesion judgment result to the display device 130, so as to help medical personnel determine whether there is a potential risk of disease through the lesion judgment result displayed on the display device 130.

Specifically, in this embodiment, the X-ray sensor 110 may have the ability to execute image subtraction, for example, logarithmic subtraction. The dual-energy image data may be obtained by performing a single exposure of the X-ray sensor 110. In this regard, the X-ray sensor 110 may, for example, have a three-layer panel structure, that is, a first sensing panel (with a first sensing array), a first metal plate (e.g., suitable metal materials such as a copper plate and lead foil), a second sensing panel (with a second sensing array), a second metal plate (e.g., suitable metal materials such as a copper plate and lead foil), and a third sensing panel (with a third sensing array) that are stacked sequentially along the sensing direction. The first metal plate and the second metal plate have the functions of absorbing energy and reducing the scattering of the sensed light. In this regard, during the exposure process of a single X-ray exposure source, the first sensing panel may capture a first initial image corresponding to a complete energy spectrum. Next, since the second sensing panel is separated from the first sensing panel by the first metal plate, and the first metal layer may absorb a portion of the X-ray energy, compared with the sensing results of the first sensing panel, the second sensing panel may obtain a second initial image corresponding to a portion of the energy spectrum (i.e., the result of subtracting the absorption energy spectrum of the first metal plate from the complete energy spectrum). Next, since the third sensing panel is separated from the second sensing panel by the second metal plate, and the second metal layer may absorb a portion of the X-ray energy, compared with the sensing results of the second sensing panel, the third sensing panel may obtain a third initial image corresponding to another portion of the energy spectrum (i.e., the result of subtracting the absorption energy spectrum of the first metal plate and the absorption energy spectrum of the second metal plate from the complete energy spectrum). Next, the X-ray sensor 110 may perform image subtraction on the first initial image, the second initial image, and the third initial image and/or directly use its raw data to generate a standard image, a soft tissue image, and a hard tissue image. Therefore, the X-ray sensor 110 may directly output the standard image, the soft tissue image, and the hard tissue image to the processing module 121 of the computing device 120.

Alternatively, in an embodiment, the dual-energy image data may also be obtained by performing a double exposure of the X-ray sensor 110. In this regard, the X-ray sensor 110 may, for example, have a single sensing panel (sensing array). During sequential exposure by two X-ray exposure sources corresponding to different energy spectra, the sensing panel of the X-ray sensor 110 may sequentially capture a high energy image corresponding to a high energy spectrum and a low energy image corresponding to a low energy spectrum. Next, the X-ray sensor 110 may perform image subtraction on the high energy image and the low energy image (i.e., dual-energy image) to generate a standard image, a soft tissue image, and a hard tissue image. Therefore, the X-ray sensor 110 may directly output the standard image, the soft tissue image, and the hard tissue image to the processing module 121 of the computing device 120.

Alternatively, in an embodiment, the image subtraction may also be performed by the processing module 121. The X-ray sensor 110 may output the dual-energy image data captured by one exposure or two exposures to the processing module 121 of the computing device 120. The processing module 121 may perform image subtraction on the dual-energy image to generate at least two of a standard image, a soft tissue image, and a hard tissue image.

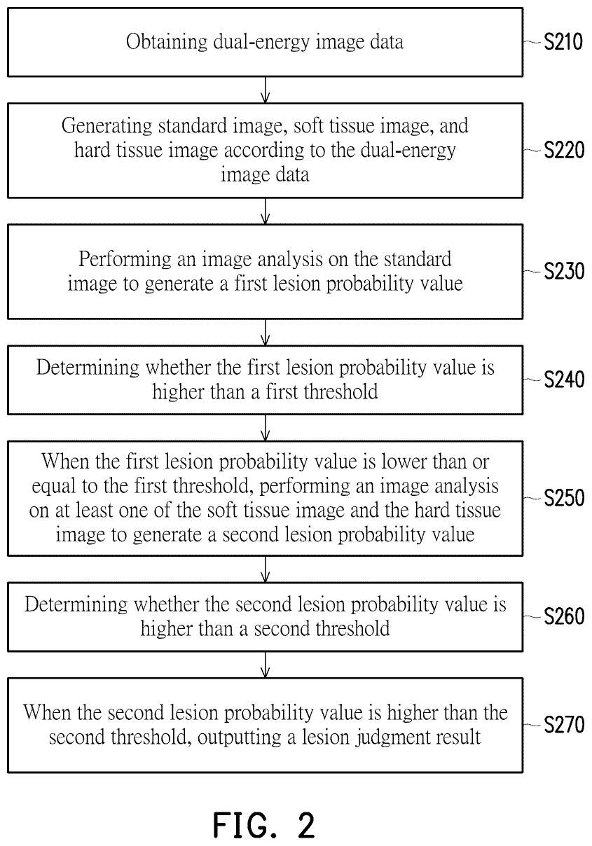

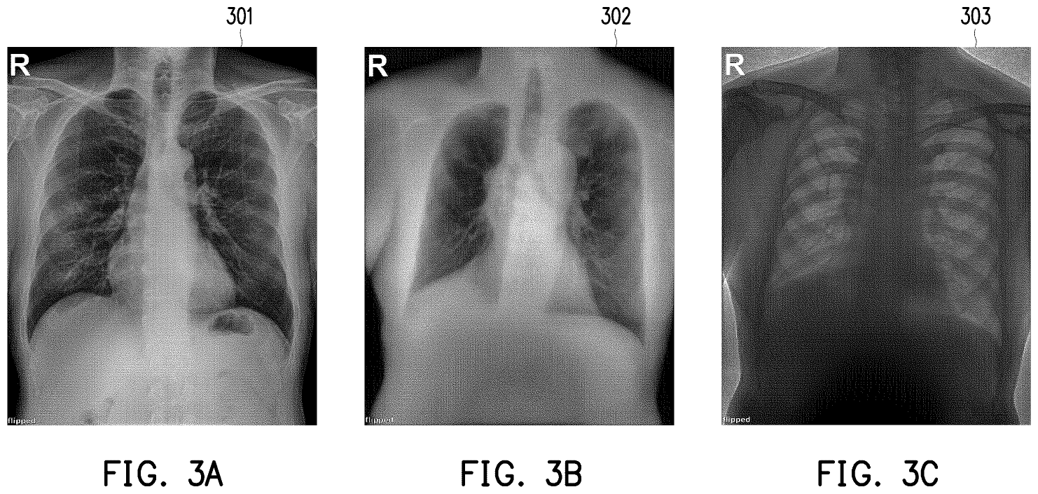

FIG. 2 is a flowchart of an image analysis method according to an embodiment of the disclosure. FIG. 3A is a schematic diagram of a standard image according to an embodiment of the disclosure. FIG. 3B is a schematic diagram of a soft tissue image according to an embodiment of the disclosure. FIG. 3C is a schematic diagram of a hard tissue image according to an embodiment of the disclosure. Referring to FIG. 1 to FIG. 3C, the image analysis system 100 may execute the image analysis method as follows in steps S210 to S270. It should be noted that the following embodiments take the sensing result of the dual-energy image of the lung as an example, but the sensing object of the X-ray sensor 110 in the disclosure is not limited to the lung.

In step S210, the X-ray sensor 110 may obtain dual-energy image data. In step S220, the X-ray sensor 110 or the processing module 121 of the computing device 120 may generate a standard image 301 corresponding to the lung region as shown in FIG. 3A, a soft tissue image 302 corresponding to the lung region as shown in FIG. 3B, and a hard tissue image 303 corresponding to the lung region as shown in FIG. 3C according to the dual-energy image data. In step S230, the processing module 121 may perform image analysis on the standard image 301 to generate a first lesion probability value.

It should be noted that the image analysis described in the various embodiments of the disclosure refers to the analysis operation performed by the image analysis unit 122_2. The input data of the image analysis may be the image data after image preprocessing, and the output data of the image analysis may be the lesion probability value output by the neural network model. In this embodiment, the processing module 121 may execute the image processing unit 122_1 to first perform image preprocessing on the standard image 301. Next, the processing module 121 may execute the image analysis unit 122_2 to perform image analysis on the standard image after image preprocessing. In this embodiment, the processing module 121 can, for example, execute a neural network model to implement image analysis, in which the neural network model may be, for example, a convolutional neural network (CNN). The neural network model may be a classification model, and may determine whether there is a lesion image in the region corresponding to the lung region in the standard image 301, so as to output the lesion probability value (i.e., the first probability value or the second probability value mentioned in each embodiment of the disclosure). The range of the lesion probability value may be represented by 0 to 1 (or represented by 0% to 100%).

It should be noted that the image preprocessing described in each embodiment of the disclosure refers to the image processing unit 122_1 performing related image processing. In this regard, related image processing may, for example, include image normalization, image enhancement, image boosting, and/or image scaling. The image normalization may be, for example, converting image values from 0 to 4096 into 0 to 1. The image enhancement may, for example, include performing image processing such as contrast limited adaptive histogram equalization (CLAHE), image sharpening, and/or image blurring. The image boosting may, for example, include executing image rotation, mirroring, image cropping, image stitching, and/or image translation, etc. The image scaling may be, for example, scaling the image size from 2500×3052 to 1024×1024.

In step S240, the image analysis unit 122_2 may determine whether the first lesion probability value is higher than a first threshold. In step S250, when the first lesion probability value is lower than or equal to the first threshold, the processing module 121 may perform image analysis on at least one of the soft tissue image 302 and the hard tissue image 303 to generate a second lesion probability value. In this regard, the processing module 121 may execute the image processing unit 122_1 to first perform image preprocessing on at least one of the soft tissue image 302 and the hard tissue image 303. Next, the processing module 121 may execute the image analysis unit 122_2 to perform image analysis on at least one of the soft tissue image 302 after image preprocessing and the hard tissue image 303 after image preprocessing, and output the second lesion probability value. In addition, when the first lesion probability value is higher than the first threshold, the processing module 121 may directly output a lesion judgment result.

In step S260, the image analysis unit 122_2 may determine whether the second lesion probability value is higher than a second threshold. In this embodiment, the first threshold may be equal to the second threshold, but the disclosure is not limited thereto. In an embodiment, the first threshold may be different from the second threshold. In step S270, when the second lesion probability value is higher than the second threshold, the processing module 121 may output a lesion judgment result. In this embodiment, the display device 130 may display the lesion judgment result. In contrast, when the second lesion probability value is lower than or equal to the second threshold, the processing module 121 may determine that no lesion has been detected.

It should be noted that the lesion judgment result described in the various embodiments of the disclosure may, for example, refer to superimposing a colored mask image corresponding to the lesion region or the lesion characteristic on the standard image 301 to display the standard image 301 marked with the lesion region or lesion characteristic. In contrast, when the processing module 121 determines that no lesion is detected, the display device 130 may simply display the original standard image 301. In addition, in an embodiment, the display device 130 may also display a text message to indicate whether a lesion has been detected.

Therefore, the image analysis system and the image analysis method of the present embodiment may effectively determine whether a lesion is present in the current sensing object. In the image analysis system and the image analysis method of the present embodiment, the first stage of lesion judgment may be performed through the standard image 301. If the processing module 121 does not determine that a lesion is present according to the standard image 301, the processing module 121 may further perform a second stage of lesion judgment according to at least one of the soft tissue image 302 and the hard tissue image 303. Therefore, the image analysis system and the image analysis method of the present embodiment may perform accurate lesion judgment on medical images.

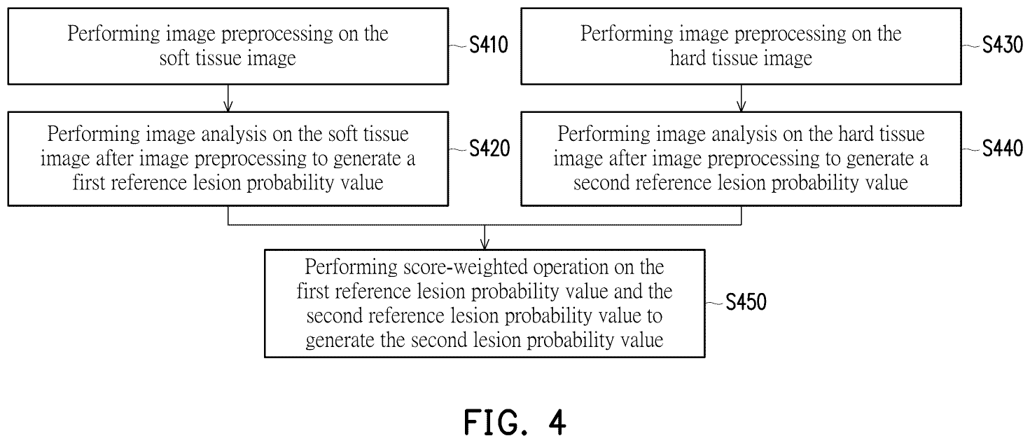

FIG. 4 is a flowchart of generating a second lesion probability value according to an embodiment of the disclosure. Referring to FIG. 1 to FIG. 4, in this embodiment, the method of generating the second lesion probability in step S250 of the embodiment in FIG. 2 may be implemented in the manner of the following steps S410 to S450. In step S410, the processing module 121 may perform image preprocessing on the soft tissue image 302. In step S420, the processing module 121 may perform image analysis on the soft tissue image after image preprocessing to generate a first reference lesion probability value. In step S430, the processing module 121 may perform image preprocessing on the hard tissue image 303. In step S440, the processing module 121 may perform image analysis on the hard tissue image after image preprocessing to generate a second reference lesion probability value. In step S450, the processing module 121 may perform a score-weighted operation on the first reference lesion probability value and the second reference lesion probability value to generate a second lesion probability value. That is to say, the processing module 121 of this embodiment may determine the second lesion probability value according to the first reference lesion probability value and the second reference lesion probability value that respectively consider the soft tissue image 302 and the hard tissue image 303, so as to effectively determine whether a lesion has occurred. However, in one embodiment, the processing module 121 may also use only the lesion probability value of the soft tissue image 302 or the hard tissue image 303 as the second lesion probability value to make a judgment.

In this embodiment, the processing module 121 may perform a score-weighted operation by adding the product of the first reference lesion probability value multiplied by the first weighting coefficient with the product of the second reference lesion probability value multiplied by the second weighting coefficient, in order to generate a second lesion probability value. For example, the processing module 121 may perform a score-weighted operation as shown in the following Formula (1). In the following Formula (1), R1 is the first reference lesion probability value. R2 is the second reference lesion probability value. α is the first weighting coefficient. β is the second weighting coefficient. R is the second lesion probability value. The first weighting coefficient α and the second weighting coefficient β are respectively between 0 and 1. In this embodiment, the sum of the first weighting coefficient α and the second weighting coefficient β is equal to 1.

$$R 1 \times α + R 2 \times β = R \qquad Formula ( 1 )$$

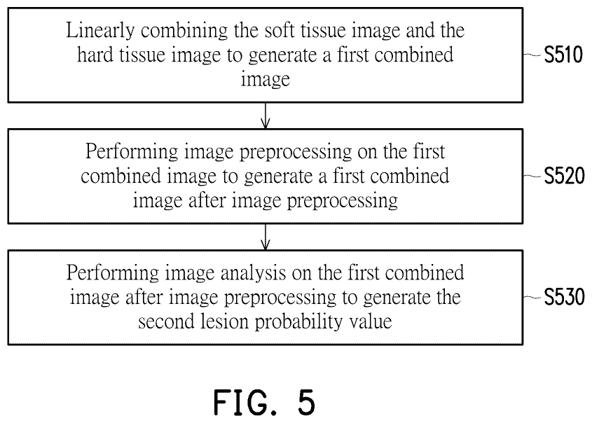

FIG. 5 is a flowchart of generating a second lesion probability value according to another embodiment of the disclosure. Referring to FIG. 1 to FIG. 3 and FIG. 5, in this embodiment, the method of generating the second lesion probability in step S250 of the embodiment in FIG. 2 may be implemented in the manner of the following steps S510 to S530. In step S510, the processing module 121 may linearly combine the soft tissue image and the hard tissue image to generate a first combined image. In this embodiment, the processing module 121 may execute image linear combination by adding the product of the pixel value of each pixel of the soft tissue image 302 multiplied by the first combination coefficient with the product of the corresponding pixel value of each pixel of the hard tissue image 303 multiplied by the second combination coefficient, in order to obtain the combined image.

It should be noted that the image linear combination described in each embodiment of the disclosure refers to the processing module 121 executing a linear combination as shown in the following Formula (2) on the pixel value of each pixel in the first image (e.g., the soft tissue image 302) and the corresponding pixel value of each pixel in the second image (e.g., the hard tissue image 303). In the following Formula (2), P1 is the pixel value of one pixel of the first image (e.g., the soft tissue image 302). P2 is a pixel value of a pixel (the position in the image corresponds to the position of the pixel of the first image) of the second image (e.g., the hard tissue image 303). Wherein a is the first combination coefficient, and b is the second combination coefficient. P is a pixel value corresponding to a pixel in the combined image. In this embodiment, the first combination coefficient α and the second combination coefficient b are respectively between 0 and 1. However, in one embodiment, if the lesion is more obvious on the first image (e.g., the soft tissue image 302), the first combination coefficient may be designed to be higher than the second combination coefficient. In addition, the image linear combination of three images may also be implemented by analogy with a formula having three combination coefficients, therefor details are not repeated herein.

$$P 1 \times a + P 2 \times b = P \qquad Formula ( 2 )$$

In step S520, the processing module 121 may perform image preprocessing on the combined image to generate a combined image after image preprocessing. In step S530, the processing module 121 may perform image analysis on the combined image after image preprocessing to generate a second lesion probability value. That is to say, the processing module 121 of this embodiment may first perform image linear combination (image superimposition) on the soft tissue image 302 and the hard tissue image 303, and the processing module 121 may then determine whether a lesion occurs on the combined images.

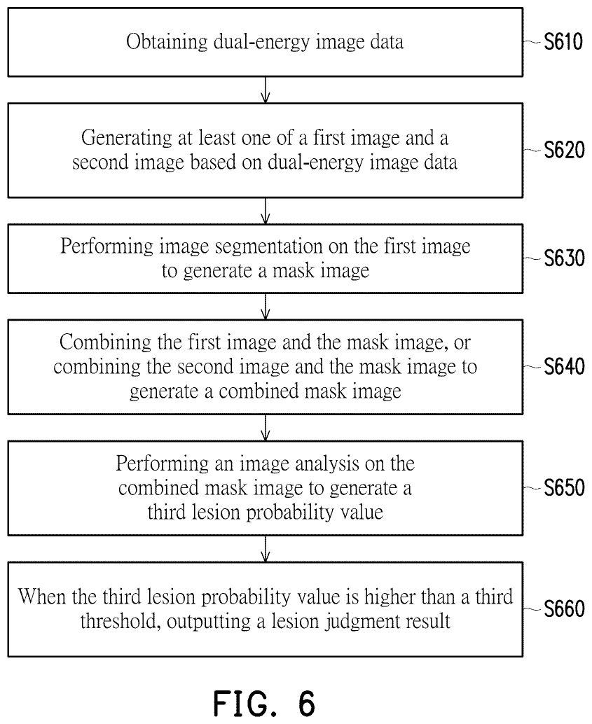

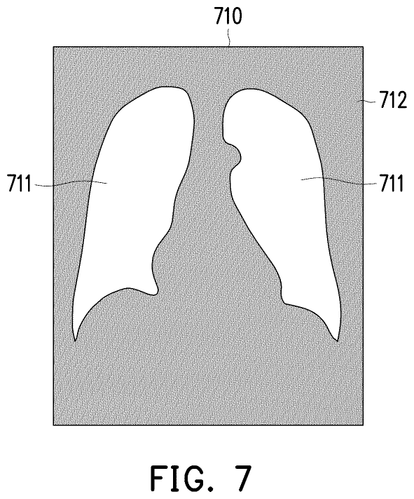

FIG. 6 is a flowchart of an image analysis method according to another embodiment of the disclosure. FIG. 7 is a schematic diagram of a mask image according to an embodiment of the disclosure. Referring to FIG. 1, FIG. 3A to FIG. 3C, and FIG. 6 to FIG. 7, the image analysis system 100 may execute the image analysis method as follows in steps S610 to S660. It should be noted that the following embodiments take the sensing result of the dual-energy image of the lung as an example, but the sensing object of the X-ray sensor 110 in the disclosure is not limited to the lung.

In step S610, the X-ray sensor 110 may obtain dual-energy image data. In step S620, the X-ray sensor 110 or the processing module 121 of the computing device 120 may generate at least one of the first image and the second image according to the dual-energy image data. In this embodiment, the X-ray sensor 110 may have the ability to perform image subtraction, so as to perform image subtraction on the dual-energy image, and output the standard image 301 as shown in FIG. 3A, the soft tissue image 302 as shown in FIG. 3B, and the hard tissue image 303 as shown in FIG. 3C to the processing module 121 of the computing device 120. Alternatively, the X-ray sensor 110 outputs the dual-energy image to the processing module 121 of the computing device 120, and the processing module 121 of the computing device 120 executes image subtraction to generate a standard image 301, a soft tissue image 302, and a hard tissue image 303. Next, the processing module 121 of the computing device 120 may use the standard image 301, the soft tissue image 302, or the hard tissue image 303 as the first image, and use the standard image 301, the soft tissue image 302, or the hard tissue image 303 as the second image. In some embodiments, the processing module 121 of the computing device 120 may also perform image linear combination on at least two of the standard image 301, the soft tissue image 302, and the hard tissue image 303 to generate the first image, and perform image linear combination on at least two of the standard image 301, the soft tissue image 302, and the hard tissue image 303 to generate the second image. In some embodiments, the processing module 121 of the computing device 120 may use the standard image 301, the soft tissue image 302 or the hard tissue image 303 as the first image, and perform image linear combination on at least two of the standard image 301, the soft tissue image 302, and the hard tissue image 303 to generate the second image.

In step S630, the processing module 121 may perform image segmentation on the first image to generate the mask image 710 as shown in FIG. 7. In this embodiment, before performing the image segmentation on the first image, the processing module 121 may first perform image preprocessing on the first image, so as to generate the first image after image preprocessing for image segmentation. In this embodiment, the processing module 121 may execute the image processing unit 122_1 to determine the lung image region in the first image and generate the corresponding mask image 710. In this embodiment, the processing module 121 may, for example, execute a neural network model to implement image segmentation, in which the neural network model may be, for example, a convolutional neural network. The neural network model may be a classification model, and may predict the probability value corresponding to the position of the lung for each pixel in the first image (for example, the probability ranges from 0 to 1). Next, the neural network model may determine whether the probability value of each pixel in the first image is greater than a preset threshold (the preset threshold is, for example, 0.7, but the disclosure is not limited thereto). When the probability value is greater than the preset threshold, the neural network model may define that the corresponding pixel in the mask image 710 has a value of 1, indicating that it belongs to the lung region 711. When the probability value is less than or equal to the preset threshold, the neural network model may define that the corresponding pixel in the mask image 710 has a value of 0, indicating that it belongs to the non-lung region 712. Therefore, the processing module 121 may have a mask image 710 with binarized pixel value as shown in FIG. 7.

In step S640, the processing module 121 combines the second image and the mask image 710 to generate a combined mask image. In this embodiment, before combining the second image and the mask image 710, the processing module 121 may perform image preprocessing on the second image. In this embodiment, the first image may be the same as the second image, or the first image may be different from the second image. When the first image is the same as the second image, the processing module 121 of the computing device 120 may directly use the first image after image preprocessing as the second image. When the first image is different from the second image, and the second image is not a single image (e.g., a standard image, a soft tissue image, or a hard tissue image), the processing module 121 of the computing device 120 may first perform image linear combination on at least two of the standard image 301, the soft tissue image 302, and the hard tissue image 303 to generate a second image different from the first image. In this embodiment, the processing module 121 may perform image superposition on the second image and the mask image 710 to generate a combined mask image. In this regard, the image superposition refers to multiplying the pixel value of each pixel of the second image by the value (i.e., the value 1 or the value 0) of the pixel corresponding to the mask image 710. In this way, when the processing module 121 analyzes the combined mask image, the processing module 121 may focus on performing image analysis on the image content of the lung region in the combined mask image (because the pixel value of the non-lung region in the combined mask image is 0).

In step S650, image analysis is performed on the combined mask image to generate a third lesion probability value. In this embodiment, when the first image is the same as the second image, the processing module 121 may execute the image analysis unit 122_2 to perform image analysis on the combined mask image. When the first image is different from the second image, the processing module 121 may execute the image processing unit 122_1 to perform image preprocessing on the combined mask image. Next, the processing module 121 may execute the image analysis unit 122_2 to perform image analysis on the combined mask image after image preprocessing, so as to generate a third lesion probability value.

In step S660, when the third lesion probability value is higher than the third threshold, the processing module 121 may output a lesion judgment result. In this embodiment, the third threshold may be equal to the first threshold or the second threshold, but the disclosure is not limited thereto. In an embodiment, the third threshold may be different from the first threshold and the second threshold. In this embodiment, the display device 130 may display the lesion judgment result. In contrast, when the third lesion probability value is lower than or equal to the third threshold, the processing module 121 may determine that no lesion has been detected.

Therefore, the image analysis system and the image analysis method of the present embodiment may effectively determine whether a lesion is present in the current sensing object. In the image analysis system and the image analysis method of this embodiment, the mask image 710 may be obtained by performing image segmentation on the first image generated by the image linear combination of the standard image 301, the soft tissue image 302, and the hard tissue image 303. Next, a single-stage lesion judgment is performed by using a combined mask image generated after the mask image 710 is combined with the second image. Therefore, the image analysis system and the image analysis method of this embodiment may accurately perform the lesion judgment of medical images, so that medical personnel may determine whether there is a potential risk of disease through the lesion judgment result displayed on the display device.

To sum up, the image analysis system and the image analysis method of this disclosure may perform image analysis on at least one of the standard image, the soft tissue image, and the hard tissue image generated by the dual-energy image, so as to obtain the lesion probability of the image. The image analysis system and the image analysis method of this disclosure may perform lesion judgment through a two-stage image analysis method or a single-stage image analysis method (combined with a mask image). Therefore, the image analysis system and the image analysis method of this disclosure may implement automatic and highly reliable lesion judgment function of medical images.

Finally, it should be noted that the foregoing embodiments are only used to illustrate the technical solutions of the disclosure, but not to limit the disclosure; although the disclosure has been described in detail with reference to the foregoing embodiments, persons of ordinary skill in the art should understand that the technical solutions described in the foregoing embodiments may still be modified, or parts or all of the technical features thereof may be equivalently replaced; however, these modifications or substitutions do not deviate the essence of the corresponding technical solutions from the scope of the technical solutions of the embodiments of the disclosure.

What is claimed is:

1. An image analysis method, comprising:
obtaining dual-energy image data;
generating a standard image, a soft tissue image, and a hard tissue image according to the dual-energy image data;
performing a first image analysis on the standard image to generate a first lesion probability value;
determining whether the first lesion probability value is higher than a first threshold;
performing a second image analysis on at least one of the soft tissue image and the hard tissue image when the first lesion probability value is lower than or equal to the first threshold, to generate a second lesion probability value;
determining whether the second lesion probability value is higher than a second threshold; and
outputting a lesion judgment result when the second lesion probability value is higher than the second threshold.

2. The image analysis method according to claim 1, wherein the dual-energy image data is generated by a single exposure of an X-ray sensor.

3. The image analysis method according to claim 1, wherein performing the first image analysis on the standard image comprises:
performing image preprocessing on the standard image to form image preprocessed data; and
performing the first image analysis on the image preprocessed data.

4. The image analysis method according to claim 1, wherein the first lesion probability value is a lesion probability value output by a neural network model.

5. The image analysis method according to claim 1, wherein performing the second image analysis on the at least one of the soft tissue image and the hard tissue image comprises:
respectively performing the second image analysis on the soft tissue image and the hard tissue image to generate a first reference lesion probability value and a second reference lesion probability value; and
performing a score-weighted operation on the first reference lesion probability value and the second reference lesion probability value to generate the second lesion probability value.

6. The image analysis method according to claim 5, wherein the score-weighted operation comprises adding a product of the first reference lesion probability value multiplied by a first weighting coefficient with a product of the second reference lesion probability value multiplied by a second weighting coefficient, to generate the second lesion probability value, wherein a sum of the first weighting coefficient and the second weighting coefficient is equal to 1.

7. The image analysis method according to claim 1, wherein performing the second image analysis on the at least one of the soft tissue image and the hard tissue image comprises:
performing a linear combination of the soft tissue image and the hard tissue image to generate a combined image;
performing image preprocessing on the combined image to generate a combined image after image preprocessing; and
performing the second image analysis on the combined image after image preprocessing to generate the second lesion probability value.

8. The image analysis method according to claim 7, wherein the linear combination comprises adding a product of a pixel value of each pixel of the soft tissue image multiplied by a first combination coefficient with a product of a corresponding pixel value of each pixel of the hard tissue image multiplied by a second combination coefficient, to obtain the combined image.

9. The image analysis method according to claim 8, wherein the first combination coefficient is higher than the second combination coefficient.

10. An image analysis method, comprising:
obtaining dual-energy image data;
generating at least one of a first image and a second image according to the dual-energy image data;
performing image segmentation on the first image to generate a mask image;
combining the first image and the mask image, or combining the second image and the mask image, to generate a combined mask image;
performing image analysis on the combined mask image to generate a third lesion probability value; and
outputting a lesion judgment result when the third lesion probability value is higher than a third threshold.

11. The image analysis method according to claim 10, wherein the dual-energy image data is generated by a single exposure of an X-ray sensor.

12. The image analysis method according to claim 10, further comprising:
first performing image preprocessing on the first image to generate a first image after image preprocessing for the image segmentation before performing the image segmentation on the first image.

13. The image analysis method according to claim 10, further comprising:
first performing image preprocessing on the combined mask image to generate a combined mask image after image preprocessing for the image analysis before performing the image analysis on the combined mask image.

14. The image analysis method according to claim 10, wherein at least one of the first image and the second image comprises a combination of at least two of a standard image, a soft tissue image, and a hard tissue image.

15. An image analysis system, comprising:
an X-ray sensor;
a computing device, coupled to the X-ray sensor, and the computing device comprising a processing module and a memory module; and
a display device, coupled to the computing device,
wherein the processing module executes an image processing unit and an image analysis unit stored in the memory module to perform an image analysis according to dual-energy image data generated by the X-ray sensor, and outputs a lesion judgment result to the display device.

16. The image analysis system according to claim 15, wherein the X-ray sensor performs image subtraction on the dual-energy image data to output at least two of a standard image, a soft tissue image, and a hard tissue image to the computing device.

17. The image analysis system according to claim 15, wherein the X-ray sensor outputs the dual-energy image data to the computing device, and the processing module performs image subtraction according to the dual-energy image data to generate at least two of a standard image, a soft tissue image, and a hard tissue image.

18. The image analysis system according to claim 15, wherein the processing module generates a standard image, a soft tissue image, and a hard tissue image according to the dual-energy image data, and the processing module performs a first image analysis on the standard image to generate a first lesion probability value,
wherein the processing module determines whether the first lesion probability value is higher than a first threshold, and the processing module performs a second image analysis on at least one of the soft tissue image and the hard tissue image when the first lesion probability value is lower than or equal to the first threshold to generate a second lesion probability value,
wherein the processing module determines whether the second lesion probability value is higher than a second threshold, and outputs a lesion judgment result when the second lesion probability value is higher than the second threshold.

19. The image analysis system according to claim 18, wherein the first lesion probability value is a lesion probability value output by a neural network model.

20. The image analysis system according to claim 15, wherein the processing module generates at least one of a first image and a second image according to the dual-energy image data, and the processing module performs image segmentation on the first image to generate a mask image,
wherein the processing module combines the first image and the mask image, or combines the second image and the mask image to generate a combined mask image, and the processing module performs an image analysis the combined mask image to generate a third lesion probability value,
wherein the processing module outputs a lesion judgment result when the third lesion probability value is higher than a third threshold.
